(19) United States
(12) Patent Application Publication
(10) Pub. No.: US 2015/0117843 A1
(43) Pub. Date: Apr. 30, 2015

(54) Fragrance-Emitting Assembly

(71) Applicant: Travis Propes, Charleston, SC (US)

(72) Inventor: Travis Propes, Charleston, SC (US)

(21) Appl. No.: 14/229,356

(22) Filed: Mar. 28, 2014

Related U.S. Application Data
(60) Provisional application No. 61/896,447, filed on Oct. 28, 2013.

(51) Int. Cl. A61L 9/03

(52) U.S. Cl. CPC A61L 9/03
USPC 392/394

(57) ABSTRACT

A fragrance-emitting apparatus (16) includes an apparatus body (14) impregnated with a fragrant fluid (12). The apparatus body (14) includes a plurality of spaced-apart support members (20) and a body member (18) formed from a porous material. The support members (20) support the body member (18) relative to a surface (10S). The body member (18) emits the fragrant fluid (12) upon heating of the support members (20). The body member (18) can include a plurality of body apertures (226). The body member (18) can have a circular or oval shape. The fragrance-emitting apparatus (16) can also include a non-porous body sealer (336) that seals at least a portion of the apparatus body (14). The body member (18) can also include one or more body features (334) that can include at least one of a body recess and a body projection.

### BACKGROUND

Individuals often desire to create a certain ambience within an environment. In some instances, such a desired ambience can be created primarily due to a fragrance, scent or aroma that is emitted within the environment. More particularly, an individual may often wish to create different ambiences, e.g., different aromas, for different situations. Thus, it is desired to provide unique, tasteful methods or devices with which such different fragrances, scents or aromas, i.e. such different ambiences, can be created. It may also be desired for such fragrances, scents or aromas to mask or cover up various foul or otherwise undesirable odors.

### SUMMARY

The present invention is directed toward a fragrance-emitting apparatus that includes a fragrant fluid and an apparatus body. In certain embodiments, the apparatus body includes a body member and a plurality of spaced-apart support members. The body member can be at least partially formed from a porous material that receives the fragrant fluid. The support members support the body member relative to a surface. In some embodiments, the body member emits the fragrant fluid upon heating of the support members.

In another embodiment, the body member is formed from a naturally porous apparatus body material. Alternatively, the body member is formed at least partially from a manmade porous apparatus body material. In certain embodiments, the body member is formed from one of a group of materials consisting of ceramic, clay brick, quarry tile, terrazzo, and concrete.

In another application of the present invention, the body member includes a plurality of body apertures that each extends through the body member. In certain embodiments, the body member can have a substantially circular or oval shape.

In one embodiment, the fragrance-emitting apparatus includes at least three spaced-apart support members. In another embodiment, the fragrance-emitting apparatus also includes a substantially non-porous body sealer that seals at least a portion of one of the support members. Further, the body sealer can seal at least a portion of the body member. The body sealer can be formed at least partially from a ceramic glaze material.

In some embodiments, the body member has a body surface, and the apparatus body includes a body recess that is formed as part of the body surface. Further, the body member can have a perimeter, and the body recess can be formed into the perimeter of the body member. In certain embodiments, the body member has a body surface, and the body member includes a plurality of body features that are formed as part of the body surface. The body features can include at least one of a body recess and a body projection.

The present invention is also directed toward a fragrance-emitting assembly that includes the fragrance-emitting apparatus and a heater that selectively heats the apparatus body so that the fragrance is emitted from the apparatus body.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified side view of one embodiment of a container holding a volume of fragrant fluid and an embodiment of a fragrance-emitting apparatus having features of the present invention;

FIG. 1B is a simplified side view of a fragrance-emitting assembly including a heater and the fragrance-emitting apparatus illustrated in FIG. 1A;

FIG. 2A is a top view of another embodiment of the fragrance-emitting apparatus;

FIG. 2B is a bottom view of the fragrance-emitting apparatus illustrated in FIG. 2A;

FIG. 2C is a side elevation view of the fragrance-emitting apparatus illustrated in FIG. 2A;

FIG. 3A is a side elevation view of still another embodiment of the fragrance-emitting apparatus;

FIG. 3B is a cross-sectional view of the fragrance-emitting apparatus taken on line 3B-3B in FIG. 3A;

FIG. 4 is a side elevation view of another embodiment of the fragrance-emitting apparatus;

FIG. 5A is a top view of yet another embodiment of the fragrance-emitting apparatus;

FIG. 5B is a bottom view of the fragrance-emitting apparatus illustrated in FIG. 5A; and

FIG. 5C is a side elevation view of the fragrance-emitting apparatus illustrated in FIG. 5A.

### DESCRIPTION

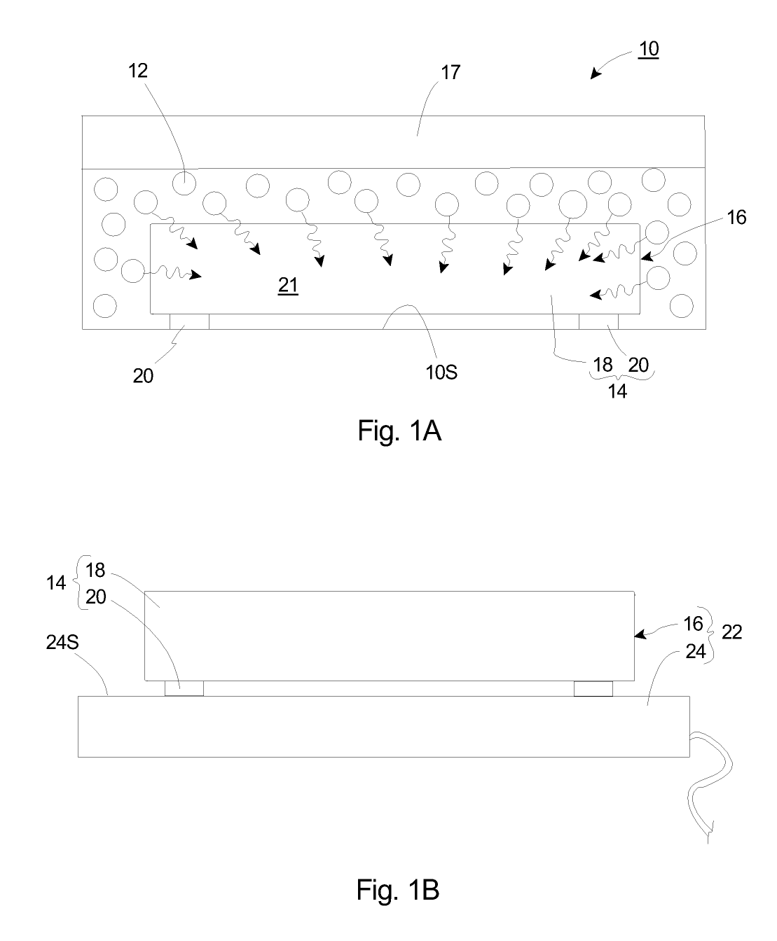

FIG. 1A is a simplified schematic side view illustration of a container 10 holding a volume of a fragrant fluid 12 (illustrated in FIG. 1A as circles, and sometimes referred to herein as a “fragrance”), and an apparatus body 14. As provided herein, in various embodiments, the apparatus body 14 is porous to allow it to soak up, be infused with or otherwise be impregnated with the fragrant fluid 12, as depicted by arrows from fragrant fluid 12 into the apparatus body 14. Once the apparatus body 14 has been impregnated with the fragrant fluid 12, the combination of the fragrant fluid 12 and the apparatus body 14 is known herein as a fragrance-emitting apparatus 16. Further, the description provided herein for FIG. 1A includes one or more steps that can be used in a method to manufacture the fragrance-emitting apparatus 16.

The fragrant fluid 12 can comprise and/or include any desired liquid or gaseous fragrance(s) that the user wishes to produce in order to create any desired ambience or other effect. For example, the fragrant fluid 12 can comprise and/or include any single desired fragrance or any desired combination of fragrances. As used herein, the term “fragrance” can include any scent, aroma, odor or other substance that can be detected by olfactory perception.

In the embodiment illustrated in FIG. 1A, the apparatus body 14 can be impregnated with the fragrant fluid 12 by soaking the apparatus body 14 in the fragrant fluid 12 that is held within the container 10. It should be appreciated that if the fragrant fluid 12 is in a gaseous form, the container 10 may include a lid 17 or suitable cover to better contain the fragrant fluid 12, in order to better impregnate the fragrant fluid 12 into the apparatus body 14. Additionally, it is understood that the container 10 need not include the lid 18 if the fragrant fluid 12 is in a liquid form or for other suitable reasons.

Because of the porosity of the apparatus body 14, the fragrant fluid 12 will be gradually absorbed into the apparatus body 14 via open pores along any surface of and within the apparatus body 14. In alternative embodiments, the scent and/or fragrance can be impregnated into the apparatus body 14 by means of pressure-infusing the fragrant fluid 12 in any suitable manner consistent with methods understood by those skilled in the art. Still alternatively, the fragrant fluid 12 can be absorbed into the apparatus body 14 by any other suitable method.

Additionally, when the fragrance-emitting apparatus 16 is heated, the fragrance from the fragrant fluid 12 that has been absorbed by the apparatus body 14 is emitted into the surrounding air. The apparatus body 14 of the fragrance-emitting apparatus 16 can be heated by any suitable method. It should be appreciated that in one embodiment the apparatus body 14 need only be heated only slightly, or by a relatively small percentage above ambient temperature, in order to enable the apparatus body 14 to emit the fragrance 12. For example, in certain embodiments, the apparatus body 14 can still be easily and comfortably handled by a person, i.e. with their bare hands, even after the apparatus body 14 has been heated enough to emit the fragrance, although care should always be taken when handling the apparatus body 14 after heating. Additionally, in some embodiments, a greater amount of heat applied to the apparatus body 14 can result in a greater amount of fragrance 12 being emitted by the apparatus body 14 in the same or shorter period of time, which can thus be utilized in a larger area, for example. However, in such applications, a greater amount of heat applied to the apparatus body 14 can result in the fragrance-producing capabilities of the fragrance-emitting apparatus 16 being depleted more quickly.

As noted above, to better enable the apparatus body 14 to absorb and/or retain the fragrant fluid 12, the apparatus body 14 can be of a porous nature. For example, the apparatus body 14 can be formed from any of a variety of natural rocks or stones, or any one of a variety of porous materials such as granite, basalt, marble, slate, limestone, travertine, sandstone, etc., that include a plurality of interconnected capillary channels (pores) that can act in a manner similar to a sponge by drawing in fluids (liquids or gases) over time. Additionally and/or alternatively, the apparatus body 14 can be formed from various manmade materials, provided such materials are of a sufficiently porous nature, such as ceramic, clay brick, quarry tile, terrazzo, concrete and/or any other suitable porous material. It should be appreciated that a greater degree of porosity of the apparatus body 14, i.e. bigger pores and/or a greater number of pores, can enable the apparatus body 14 to absorb the fragrant fluid 12 more quickly and also to emit the desired scent or aroma more easily (i.e. with less heating required).

Further, the apparatus body 14 can be reused as desired. For example, when the fragrant fluid 12 has substantially all been emitted from the apparatus body 14, the apparatus body 14 can be again infused, soaked or otherwise impregnated with the fragrant fluid 12 that can have a same or a different fragrance from the previously used fragrant fluid 12.

In certain embodiments, the apparatus body 14 can include a body member 18 and one or more support members 20 that support the body member 18 above any surface, such as a bottom surface 10S of the container 10, or other surfaces such as a table, a countertop, a support plate, a bookshelf, a desk, or any other surface including but not limited to, those described herein. In the embodiment illustrated in FIG. 1A, the body member 18 includes a body surface 21. The body surface 21 includes any exterior surface of the body member 18.

FIG. 1B is a simplified side view of a fragrance-emitting assembly 22 including a heater 24 and the fragrance-emitting apparatus 16 illustrated in FIG. 1A. As noted above, the fragrance-emitting apparatus 16 can be heated by any suitable heating apparatus or method as a means to more effectively enable the fragrance-emitting apparatus 16 to emit the desired fragrance. For example, as illustrated in FIG. 1B, the fragrance-emitting apparatus 16 can be heated by simply positioning the fragrance-emitting apparatus 16 on or near any suitable surface of the heater 24.

In one embodiment, the heater 24 can be an “on/off” type of heater that does not have an adjustable temperature setting. In another embodiment, the heater 24 can have an adjustable heat setting (not shown) that allows the user to adjust the temperature of the heater 24 when turned on. In certain embodiments, the temperature of the heater 24 required to cause the fragrance 12 of in the fragrance-emitting apparatus to begin to be emitted can be relatively low. For example, in certain non-exclusive embodiments, the heater 24 can have a temperature setting of one or more of: approximately 200 degrees F., approximately 175 degrees F., approximately 150 degrees F., approximately 140 degrees F., approximately 130 degrees F., approximately 120 degrees F., approximately 110 degrees F. or approximately 100 degrees F. In other non-exclusive embodiments, the heater 24 can have a temperature setting of greater than approximately 200 degrees F.

Additionally, or in the alternative, in certain embodiments, the heater 24 can have a timer (not shown) that allows the heater 24 to automatically turn on or off either at a designated time, or for a designated period of time. For example, in one embodiment, the heater 24 can be set by a user to turn on at a particular time of day, remain on for a specific amount of time, and then automatically shut off. Alternatively, the user can manually turn on the heater 24, which then stays on for a certain period of time before automatically shutting off.

In the embodiment illustrated in FIG. 1B, the support members 20 support the body member above a surface 24S of the heater. In this embodiment, the heater 24 can heat the support members 20, which in turn can heat the body member 18, which then causes the desired fragrance 12 to be emitted. Alternatively, the apparatus body 14 can omit the support members 20, and the body member 18 can rest directly on the surface 24S of the heater 24, or any other suitable surface.

Heating of the support member(s) 20 causes a transfer of heat via thermal conduction to the body member 18. However, because the body member 18 is not placed directly onto the heater 24, it is easier for a user to touch and/or handle the body member 18 even after heating because the body member 18 is indirectly heated via the support members 20. Thus, the body member 18 is not necessarily heated to the same temperature as the support members 20, thereby increasing the safety of directly handling the body member 18.

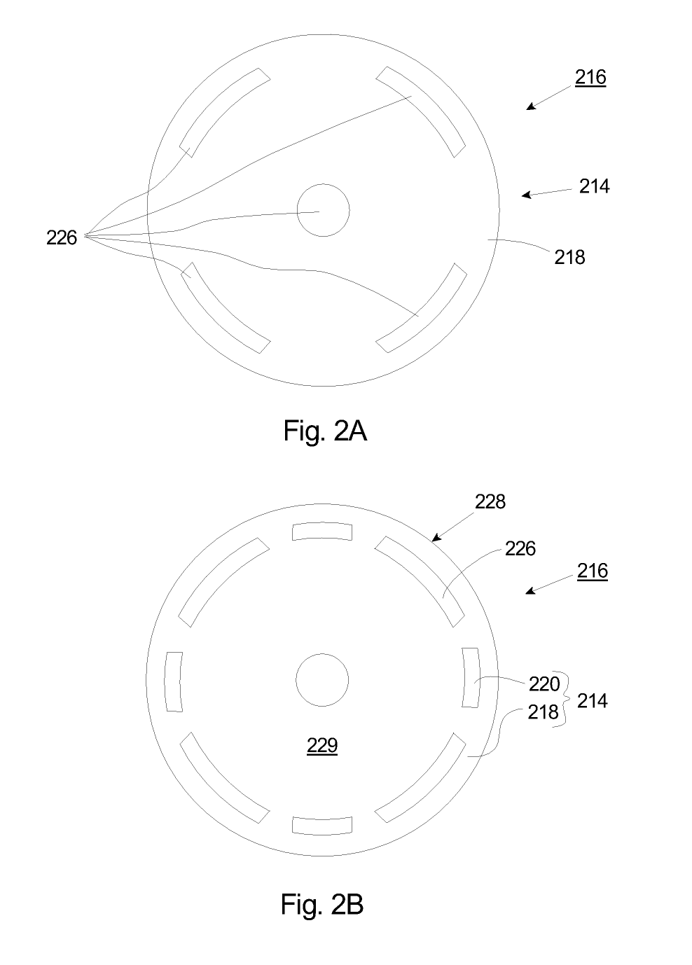

FIG. 2A is a top view of another embodiment of the fragrance-emitting apparatus 216 which includes the apparatus body 214 impregnated with fragrant fluid 12 (illustrated in FIG. 1A). In the embodiment illustrated in FIG. 2A, the apparatus body 214 includes a body member 218 and a plurality of body apertures 226. In one embodiment, the body member 218 includes one or more of the body apertures 226. In this embodiment, the body member 218 has a substantially circular shape. It is recognized, however, that the body member 218 can have a different configuration. For example, the body member 218 can be rectangular disk-shaped, square disk-shaped, triangular disk-shaped, hexagon disk-shaped, octagon disk-shaped, oval disk-shaped, or some other form of disk-shape. Still alternatively, the body member 218 need not be disk-shaped. For example, in certain non-exclusive alternative embodiments, the body member 218 can be substantially spherical, rectangular, cube-shaped, pyramid-shaped, cone-shaped, or any other suitable shape.

In the embodiment illustrated in FIG. 2A, the body member 218 includes five body apertures 226. The body apertures 226 can provide one or more functions, including (i) increasing a surface area of the body member 218 to facilitate and expedite the fragrance impregnation process; (ii) increasing a surface area of the body member 218 to increase the rate at which the fragrance is emitted from the body member 218; (iii) providing an aesthetic or decorative look to the body member 218; and/or (iv) providing safer and easier handling by the user in the form of handholds on the apparatus body 214, among other functions.

In one embodiment, the body apertures 226 can have varying configurations. For example, in the embodiment illustrated in FIG. 2A, four of the body apertures 226 have a somewhat rectangular/arc-shaped configuration, and one of the body apertures 226 is somewhat circular in shape. However, it is understood that the body member 218 can have any number of body apertures 226, and the body apertures 226 can have any suitable shape or configuration. For example, in certain embodiments, one or more of the body apertures 226 can be round, oval, square, semi-circular, trapezoidal or hexagonal, or one or more of the body apertures 226 can have any other polygonal or curved shape, as non-exclusive examples. Still alternatively, one or more of the body apertures 226 can have a different shape or configuration. Further, the positioning of the body apertures 226 can vary to suit the design or aesthetic requirements of the body member 218.

FIG. 2B is a bottom view of the fragrance-emitting apparatus 216 illustrated in FIG. 2A. In the embodiment illustrated in FIG. 2B, the body apertures 226 are illustrated. Further, in this embodiment, the apparatus body 214 includes a plurality of support members 220. For example, in the embodiment illustrated in FIG. 2B, the apparatus body 214 includes four rectangular, slightly arc-shaped support members 220 that are substantially evenly spaced and positioned near a circumference 228 of the body member 218. However, it is recognized that there can be any suitable number of support members 220 (greater or fewer than four, for example), the support members 220 can have any suitable shape or configuration, and the support members 220 can be positioned in any suitable location on the body member 218. For example, in one non-exclusive alternative embodiment, the support members 220 can be somewhat hemi-spherical in shape so that each support member 220 provides a single point of contact with the surface 210S (illustrated in FIG. 2C, for example). Stated another way, with this design, because less of the apparatus body 214 is in contact with the surface 210S, more of the apparatus body 214 is in contact with the fragrant fluid 12 during the impregnation process. Further, more of the apparatus body 214 is exposed to the air for greater emission of the fragrance 12 during use.

In one embodiment, one or more of the support members 220 can be integrally formed with the body member 218 to form the apparatus body 214. Stated another way, one or more of the support members 220 and the body member 218 can be formed as a unitary structure. Alternatively, one or more of the support members 220 can be separately attached to the body member 218 to form the apparatus body 214.

FIG. 2C is a side elevation view of the apparatus body 214 of the fragrance-emitting apparatus 216 illustrated in FIG. 2A. As shown in FIG. 2C, the support members 220 can extend away from a bottom surface 229 of the apparatus body 214. When the apparatus body 214 is positioned on a surface 210S, the apparatus body 214 can be positioned such that the support members 220 contact and/or engage the surface 210S and maintain the apparatus body 214 spaced apart from the surface 210S. With this design, only the support members 220 are in contact with the surface 210S, and a majority of the heated apparatus body material can be maintained spaced apart from the surface 210S. Additionally, with this design, any potential damage from the heated apparatus body material being positioned adjacent to the surface 210S can be inhibited, and the desired scent or aroma can be more effectively emitted from all surfaces of the apparatus body 214.

In various embodiments, the body member 218 has a body height 230, and one or more support members 220 has a support height 232. The body height 230 and the support height 232 can vary. In one embodiment, a ratio of the body height 230 to the support height 232 can be approximately 5:1. In non-exclusive alternative embodiments, the ratio of the body height 230 to the support height 232 can be approximately 1:2, 1:1, 2:1, 3:1, 4:1, 6:1, 8:1 or 10:1. In still other embodiments, the ratio of the body height 230 to the support height 232 can be less than 1:2 or greater than 10:1.

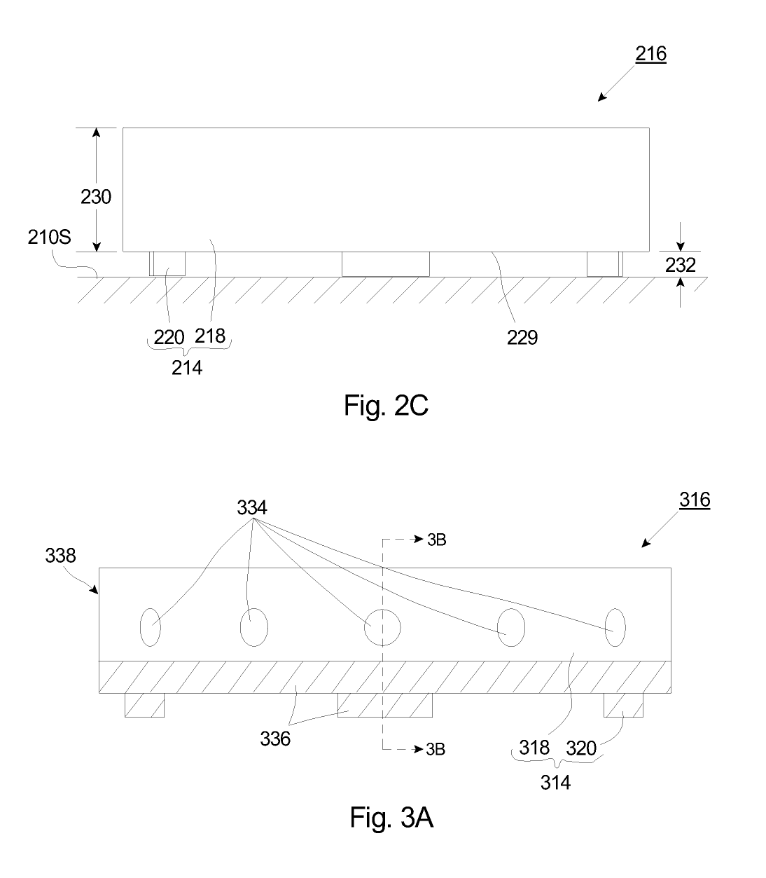

FIG. 3A is a side elevation view of another embodiment of an apparatus body 314 of a fragrance-emitting apparatus 316. In the embodiment illustrated in FIG. 3A, the apparatus body 314 includes a body member 318 and a plurality of support members 320. Additionally, in this embodiment, the apparatus body 314 can include one or more of a plurality of body features 334 and a body sealer 336. The body features 334 provide a greater surface area of the apparatus body 314 for the absorbance or impregnation of the fragrant fluid 12 (illustrated in FIG. 1A) into the apparatus body 314. Additionally or alternatively, the body features 334 can provide greater surface area for the emission of the fragrance from the apparatus body 314 and/or can aesthetically add to the overall appearance of the fragrance-emitting apparatus 316. In certain embodiments, one or more of the body features 334 can be indentations, recesses or depressions (also sometimes referred to herein as “body recesses”) into one or more of the surfaces of the apparatus body 314. Alternatively, one or more of the body features 334 can be protrusions or projections (also sometimes referred to herein as “body projections”) that protrude or extend outwardly from one or more of the surfaces of the apparatus body 314. In one embodiment, the body features 334 can include a combination of body recesses and body projections.

The size, shape, quantity and positioning of the body features 334 can be varied to suit the design requirements of the fragrance-emitting apparatus 316. In one embodiment, one or more of the body features 334 can have a circular shape. Alternatively, one or more of the body features 334 can have an oval, rectangular, or any other suitable shape, design or configuration. In one embodiment, one or more of the body features 334 can be positioned near or along a perimeter 338 of the body member 318. Additionally or alternatively, one or more of the body features 334 can be positioned on any other surface of the apparatus body 314.

The body sealer 336 can protect the apparatus body 314 from damage during direct or indirect heating by the heater 24 (illustrated in FIG. 1B). In one embodiment, the body sealer 336 (illustrated by shaded area in FIG. 3A) can be a ceramic glaze material that coats and/or seals at least a portion of the apparatus body 314. Alternatively, the body sealer 336 can include other suitable types of materials that seal or otherwise cover a portion or all of the apparatus body 314 and provide a hardened protective layer over a portion or all of the apparatus body 314. In one embodiment, the body sealer 336 can seal portions or all of one or more of the support members 320 and/or a portion or the entire body member 318. In the embodiment illustrated in FIG. 3A, the body sealer 336 seals or otherwise covers the support members 320 and a portion of the body member 318. The body sealer 336 can be decorative and/or can protect the apparatus body 314 from damage or breakage during heating of the fragrance-emitting apparatus 316.

FIG. 3B is a cross-sectional view of the fragrance-emitting apparatus 316 taken on line 3B-3B in FIG. 3A. In this embodiment, the body sealer 336 can be viewed as coating and/or sealing a portion of the apparatus body 314 including the body supports 320 and a portion of the body member 318. Further, in FIG. 3B, the body features 334 are illustrated as indentations or recesses, although as provided herein the body features 334 can alternatively be protrusions or a combination of indentations and protrusions.

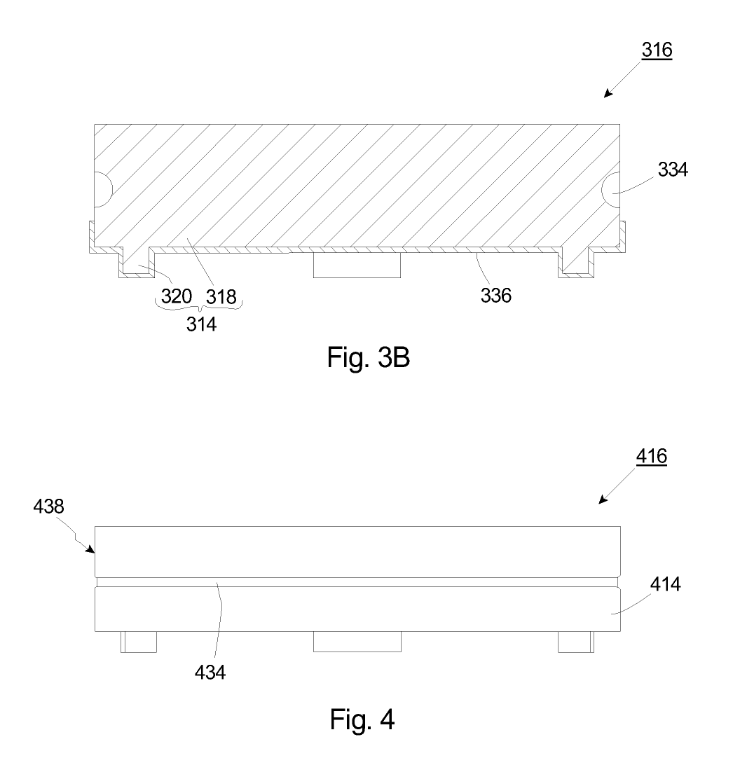

FIG. 4 is a side elevation view of another embodiment of a fragrance-emitting apparatus 416. In this embodiment, the apparatus body 414 includes a body feature 434 that provides a greater surface area of the apparatus body 414 for the absorbance or impregnation of the fragrant fluid 12 (illustrated in FIG. 1A) into the apparatus body 414. Additionally or alternatively, the body feature 434 can provide greater surface area for the emission of the fragrance from the apparatus body 414 and/or can aesthetically add to the overall appearance of the fragrance-emitting apparatus 416. In this embodiment, the body feature 434 can be an indentation or a depression into one or more of the surfaces of the apparatus body 414. In an alternative embodiment, the body feature 434 can be a projection that protrudes or extends outwardly from one or more of the surfaces of the apparatus body 414. In the embodiment illustrated in FIG. 4, the body feature 434 extends partially or fully around a perimeter 438 of the apparatus body 414. Additionally or alternatively, the body feature 434 can be positioned on any other surface of the apparatus body 414.

The shape and positioning of the body feature 434 can be varied to suit the design requirements of the fragrance-emitting apparatus 416. In one embodiment, the body feature 434 can have a circular or circumferential shape. Alternatively, the body features 434 can have an oval, rectangular, or any other suitable shape, design or configuration.

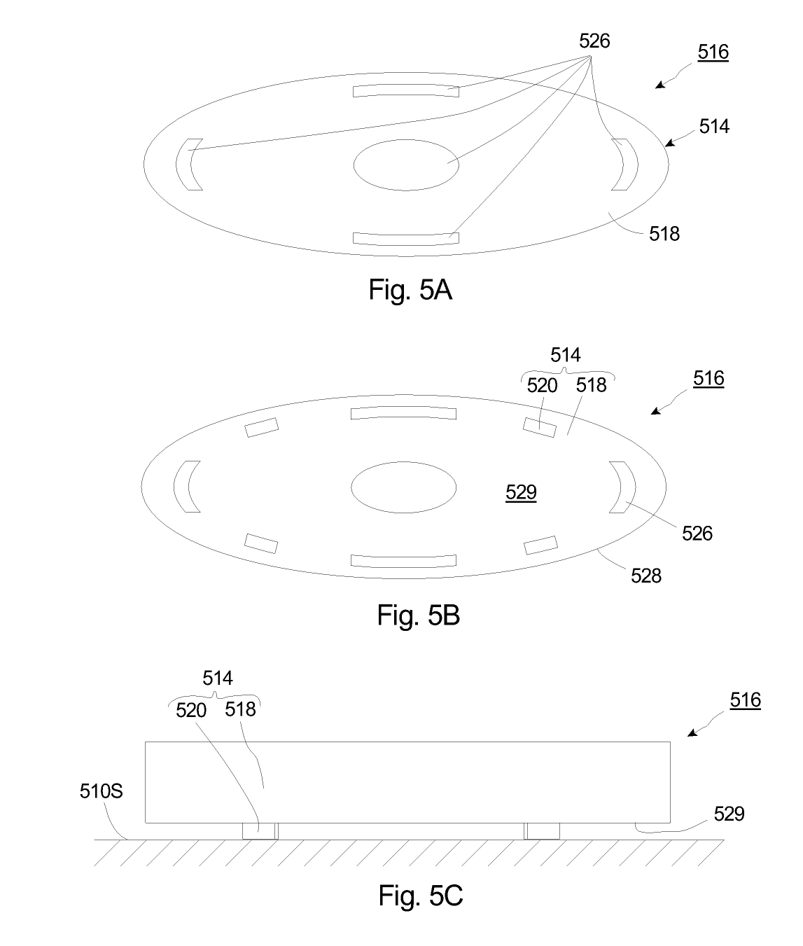

FIG. 5A is a top view of another embodiment of the fragrance-emitting apparatus 516 which includes the apparatus body 514 impregnated with fragrant fluid (not shown in FIG. 5A). In the embodiment illustrated in FIG. 5A, the apparatus body 514 includes a body member 518 and a plurality of body apertures 526. In this embodiment, the body member 518 has a substantially circular shape. It is recognized, however, that the body member 518 can have a different configuration. For example, the body member 518 can be rectangular disk-shaped, square disk-shaped, triangular disk-shaped, hexagon disk-shaped, octagon disk-shaped, oval disk-shaped, or some other form of disk-shape. Still alternatively, the body member 518 need not be disk-shaped. For example, in certain non-exclusive alternative embodiments, the body member 518 can be substantially spherical, rectangular, cube-shaped, pyramid-shaped, cone-shaped, or any other suitable shape.

In the embodiment illustrated in FIG. 5A, the body member 518 includes five body apertures 526. The body apertures 526 can provide one or more functions, including (i) increasing a surface area of the body member 518 to facilitate and expedite the fragrance impregnation process; (ii) increasing a surface area of the body member 518 to increase the rate at which the fragrance is emitted from the body member 518; (iii) providing an aesthetic or decorative look to the body member 518; and/or (iv) providing safer and easier handling by the user in the form of handholds on the apparatus body 514, among other functions.

In one embodiment, the body apertures 526 can have varying configurations. For example, in the embodiment illustrated in FIG. 5A, four of the body apertures 526 have a somewhat rectangular/arc-shaped configuration, and one of the body apertures 526 is somewhat circular in shape. However, it is understood that the body member 518 can have any number of body apertures 526, and the body apertures 526 can have any suitable shape or configuration. For example, in certain embodiments, one or more of the body apertures 526 can be round, oval, square, semi-circular, trapezoidal or hexagonal, or one or more of the body apertures 526 can have any other polygonal or curved shape, as non-exclusive examples. Still alternatively, one or more of the body apertures 526 can have a different shape or configuration. Further, the positioning of the body apertures 526 can vary to suit the design or aesthetic requirements of the body member 518.

FIG. 5B is a bottom view of the fragrance-emitting apparatus 516 illustrated in FIG. 5A. In the embodiment illustrated in FIG. 5B, the body apertures 526 are illustrated. Further, in this embodiment, the apparatus body 514 includes a plurality of support members 520. For example, in the embodiment illustrated in FIG. 5B, the apparatus body 514 includes four rectangular, slightly arc-shaped support members 520 that are substantially evenly spaced and positioned near a circumference 528 of the body member 518. However, it is recognized that there can be any suitable number of support members 520 (greater or fewer than four, for example), the support members 520 can have any suitable shape or configuration, and the support members 520 can be positioned in any suitable location on the body member 518. For example, in one non-exclusive alternative embodiment, the support members 520 can be somewhat hemi-spherical in shape so that each support member 520 provides a single point of contact with the surface 510S (illustrated in FIG. 5C, for example). Stated another way, with this design, because less of the apparatus body 514 is in contact with the surface 510S, more of the apparatus body 514 is in contact with the fragrant fluid 12 (illustrated in FIG. 1A) during the impregnation process. Further, more of the apparatus body 514 is exposed to the air for greater emission of the fragrance during use.

In one embodiment, one or more of the support members 520 can be integrally formed with the body member 518 to form the apparatus body 514. Stated another way, one or more of the support members 520 and the body member 518 can be formed as a unitary structure. Alternatively, one or more of the support members 520 can be separately attached to the body member 518 to form the apparatus body 514.

FIG. 5C is a side elevation view of the apparatus body 514 of the fragrance-emitting apparatus 516 illustrated in FIG. 5A. As shown in FIG. 5C, the support members 520 can extend away from a bottom surface 529 of the apparatus body 514. When the apparatus body 514 is positioned on a surface 510S, the apparatus body 514 can be positioned such that the support members 520 contact and/or engage the surface 510S and maintain the apparatus body 514 spaced apart from the surface 510S. With this design, only the support members 520 are in contact with the surface 510S, and a majority of the heated apparatus body material can be maintained spaced apart from the surface 510S. Additionally, with this design, any potential damage from the heated apparatus body material being positioned adjacent to the surface 510S can be inhibited, and the desired scent or aroma can be more effectively emitted from all surfaces of the apparatus body 514.

While a number of exemplary aspects and embodiments of a fragrance-emitting apparatus 16 and a fragrance-emitting assembly 22 have been discussed above, those skilled in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A fragrance-emitting apparatus comprising:
a fragrant fluid; and
an apparatus body including a body member and a plurality of spaced-apart support members, the body member being at least partially formed from a porous material that receives the fragrant fluid, the support members supporting the body member relative to a surface;
wherein the body member emits the fragrant fluid upon heating of the support members.

2. The fragrance-emitting apparatus of claim 1 wherein the body member is formed from a naturally porous apparatus body material.

3. The fragrance-emitting apparatus of claim 1 wherein the body member is formed at least partially from a manmade porous apparatus body material.

4. The fragrance-emitting apparatus of claim 1 wherein the body member is formed from one of a group of materials consisting of ceramic, clay brick, quarry tile, terrazzo, and concrete.

5. The fragrance-emitting apparatus of claim 1 wherein the body member includes a plurality of body apertures that each extends through the body member.

6. The fragrance-emitting apparatus of claim 1 wherein the body member has a substantially oval shape.

7. The fragrance-emitting apparatus of claim 1 wherein the fragrance-emitting apparatus includes at least three spaced-apart support members.

8. The fragrance-emitting apparatus of claim 1 further comprising a substantially non-porous body sealer that seals at least a portion of one of the support members.

9. The fragrance-emitting apparatus of claim 8 wherein the body sealer seals at least a portion of the body member.

10. The fragrance-emitting apparatus of claim 8 wherein the body sealer is formed at least partially from a ceramic glaze material.

11. The fragrance-emitting apparatus of claim 1 wherein the body member has a body surface, the apparatus body including a body recess that is formed as part of the body surface.

12. The fragrance-emitting apparatus of claim 11 wherein the body member has a perimeter, and the body recess is formed into the perimeter of the body member.

13. The fragrance-emitting apparatus of claim 1 wherein the body member has a body surface, the body member including a plurality of body features that are formed as part of the body surface, the body features including at least one of a body recess and a body projection.

14. A fragrance-emitting assembly comprising the fragrance-emitting apparatus of claim 1 and a heater that selectively heats the apparatus body so that the fragrance is emitted from the apparatus body.

15. A fragrance-emitting apparatus comprising:
a fragrant fluid; and
a body member that is at least partially formed from a porous material, the body member receiving the fragrant fluid, the body member having a body surface, the body member including a body recess that is formed as part of the body surface;
wherein the body member emits the fragrant fluid upon heating of the body member.

16. The fragrance-emitting apparatus of claim 15 further comprising a plurality of support members that support the body member relative to a surface.

17. The fragrance-emitting apparatus of claim 16 wherein the support members and the body member are formed as a unitary structure.

18. The fragrance-emitting apparatus of claim 15 wherein the body member is formed from one of a group of materials consisting of ceramic, clay brick, quarry tile, terrazzo, and concrete.

19. The fragrance-emitting apparatus of claim 15 further comprising (i) a plurality of support members that support the body member relative to a surface, and (ii) a substantially non-porous sealer that seals at least a portion of one of the support members.

20. A fragrance-emitting assembly comprising the fragrance-emitting apparatus of claim 15 and a heater that selectively heats the body member so that the fragrance is emitted from the body member.

21. A fragrance-emitting apparatus comprising:
a fragrant fluid;
a body member that is at least partially formed from a porous material, the body member receiving the fragrant fluid, the body member having a body surface, the body member including a body recess that is formed as part of the body surface; and
a plurality of spaced-apart support members that support the body member relative to a surface;
wherein the apparatus body emits the fragrant fluid upon heating of the body member.

22. The fragrance-emitting apparatus of claim 21 further comprising a substantially non-porous sealer that seals at least a portion of one of the plurality of support members.
